(19) United States
(12) Patent Application Publication
(10) Pub. No.: US 2022/0333362 A1
(43) Pub. Date: Oct. 20, 2022

(54) MEDICAL WASTE DRAIN SYSTEM

(71) Applicant: Charles Watson, Reynoldsburg, OH (US)

(72) Inventor: Charles Watson, Reynoldsburg, OH (US)

(21) Appl. No.: 17/230,043

(22) Filed: Apr. 14, 2021

(51) Int. Cl. E03C 1/284

(52) U.S. Cl. CPC E03C 1/284; A61M 1/14

(57) ABSTRACT

A system in a residential setting that carries dialysis waste to a sewer line safely, so as not to contaminate other areas, and is easily removed when dialysis is no longer needed in the residence.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### FIELD OF THE TECHNOLOGY

The subject technology is in the technical field of medical waste removal into municipal sewer systems, particularly in a residential setting.

### BACKGROUND OF THE TECHNOLOGY

Dialysis is, of course, the replacement or imitation of kidney processes to remove excess water and waste from the blood, so as to facilitate the body to produce red blood cells. When kidneys fail to do this, then the process must be done by other means. In the past, persons needing dialysis would have to travel to a dialysis center periodically, to have this service performed. Recent technological advances now enable persons who need this assistance to have it done in their homes or other residential setting; sometimes an assistant is needed, and sometimes the person requires no assistance. However, dialysis in a residential setting requires the proper equipment and infrastructure.

In addition to proper equipment, power, water, chemical solutions, communication in case assistance is needed, lighting, and comfortable furniture, a drainage system is required to dispose of the waste and excess water that the home dialysis process removes. Such waste may be properly and legally disposed into the municipal sewer system.

Several drainage systems have been, and are currently used for this. All involve a drainage line to carry the waste away from a source, and to a portal into which the waste is conveyed into the sewer system. Most, if not all, interface with a system that allows water to flow to assist disposal, such as use in conjunction with a sink or toilet. Consider first a tundish, which is a funnel fining installed on pipes into which the drainage line is placed. Consider also systems such as disclosed by DiMaggio in U.S. Pat. No. 7,090,179, Mounting Assembly For Waste Line of a Medical Treatment Apparatus, and other references in that family of applications by DiMaggio. Here, the drainage line brings waste into a sink or toilet.

In all of the above, water via faucet or toilet flush can be used to help move the waste down the drain. Also, in all of the above, the drainage systems rely on gravity, air pressure, weight, and water seeking its own level to move the waste. No additional pumping or other force is applied from the dialysis machine. This makes such disposal different from forced disposal into the sewer system, such as from a dishwasher, as disclosed by Vila in U.S. Pat. No. 6,588,803, Extendable P-Trap Dishwasher Waste Port. Waste water from a dishwasher may alternately be channeled through a garbage disposal, or directly into pipes just below a sink through use of a tailpiece with side arm. A problem with the tailpiece and side arm for dialysis waste removal is that waste water may splash up into the sink. A problem with conducting dialysis waste through a garbage disposal is that waste water may splash up into the sink when the garbage disposal is used. A problem with the tailpiece and side arm for dialysis waste removal is that waste water may splash up into the sink.

### NEED FOR SUBJECT TECHNOLOGY

What is needed is a system in a residential setting that carries dialysis waste to a sewer line safely, so as not to contaminate other areas, and is easily removed when dialysis is no longer needed in the residence.

### SUMMARY OF THE TECHNOLOGY

The subject technology is a system and a method for its use. The system includes a conduit carrying dialysis waste from a source to a plumbing trap specifically designed for quick connection to the conduit, and adapted to receive dialysis waste that is delivered via gravity, air pressure, weight, and water seeking its own level to move the waste.

### BRIEF DESCRIPTION OF THE DRAWINGS

FIG. 1A shows a side view of the plumbing trap.

FIG. 1B is a perspective view of the plumbing trap.

FIG. 1C is a view of a conduit and distal end.

FIGS. 2A, 2B, and 2C show an adaptor currently used for connection.

FIG. 3A shows the subject technology in a typical residential setting.

FIG. 3B is a close-up view showing connections.

FIG. 4A and 4B show flow charts of the installation and removal methods, respectively.

### DETAILED DESCRIPTION OF THE TECHNOLOGY

The subject technology will be described more fully with reference to the accompanying drawings, in which a preferred embodiment of the subject technology is shown. However, persons of ordinary skill in the appropriate arts may modify the subject technology described here while still achieving the favorable results. Accordingly, the description which follows is to be understood as being a broad, teaching disclosure directed to persons of ordinary skill in the appropriate arts, and not as limiting upon the subject technology.

Certain definitions are used herein:

“Clearing” is a process of removing liquid that may remain in a conduit, forcing the liquid out generally by raising one end of the conduit and intermediate sections of the conduit while the other end delivered liquid to a container or a drainage system.

A “conduit” is a drainage line, or “drain line,” that carries dialysis liquid waste from a source such as a dialysis machine, to a destination, for disposal into a municipal sewer system.

“Dialysis tubing” are the paired connections to a patient's artery and vein, typically in the patient's arm, for delivering blood from the artery to the dialysis machine and returning cleansed blood to patient's vein.

A “plumbing trap” is a drainpipe fixture between a sink's drain and a municipal sewer system, provided with a curve or bend which holds water as a barrier to prevent sewer gasses from entering through the sink. Liquid poured into the sink passes through the plumbing trap, leaving an amount of liquid in the curved portion to create the barrier. Common implementations for plumbing traps are called “J bends” and “P traps.”

An “inlet” is a side portal into a plumbing fixture, such as a drainpipe or a plumbing trap, providing an alternative entry for liquid.

A “quick connection” is a device for connecting a conduit to an inlet.

### REFERENCE NUMBERS

The following reference numbers are used throughout the drawings and this specification.
100 system
101 dialysis machine
102 conduit
104 proximal end
106 distal end
108 first quick connection
110 plumbing trap
112 angled inlet
114 receiving end
116 second quick connection
120 dialysis tubing
122 water source
123 water line
124 sink
200 install method
202 removing
204 installing
206 connecting
208 removing
220 removal method
222 disconnecting
224 clearing
230 installing

### DETAILED DESCRIPTION OF THE DRAWINGS AND BEST MODE OF THE PREFERRED EMBODIMENT

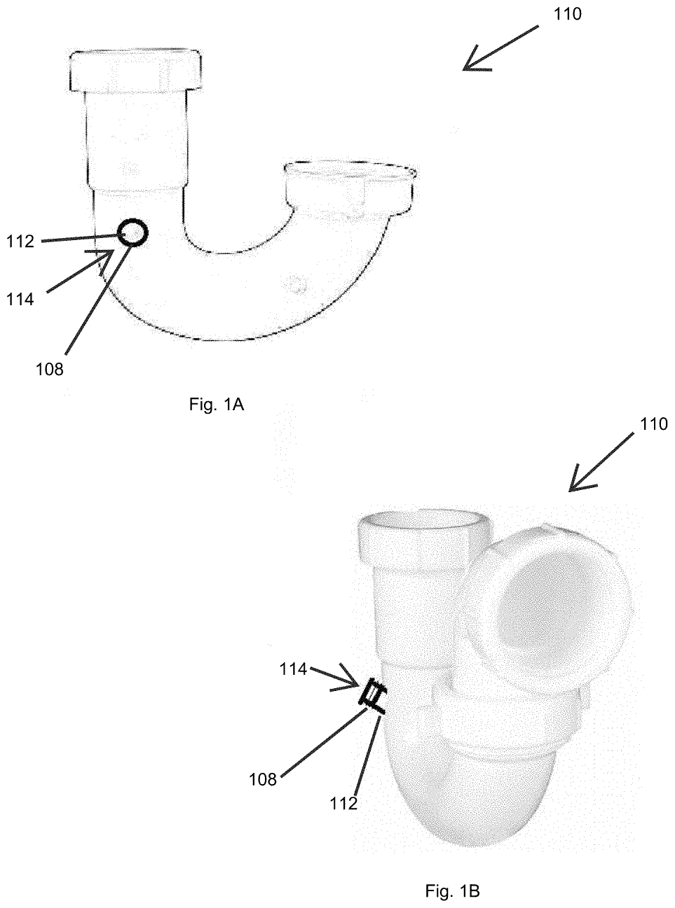

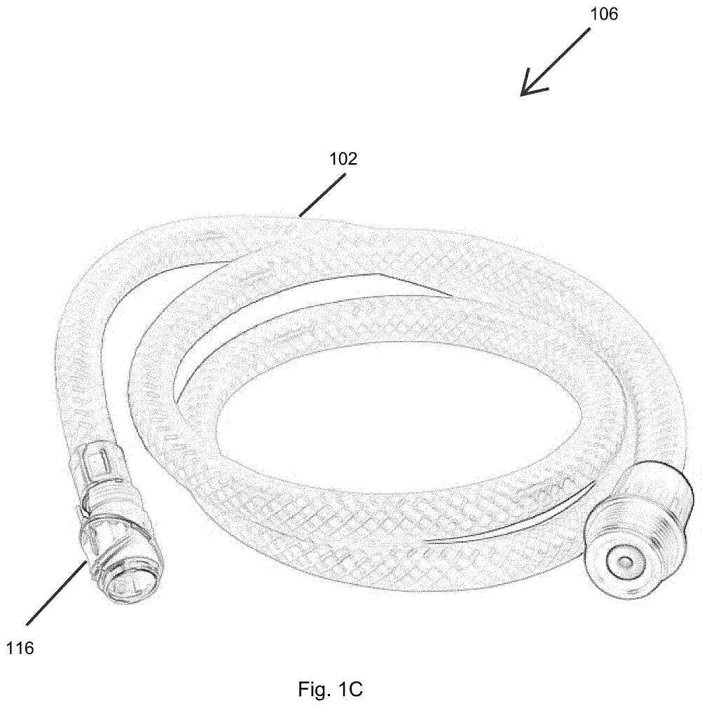

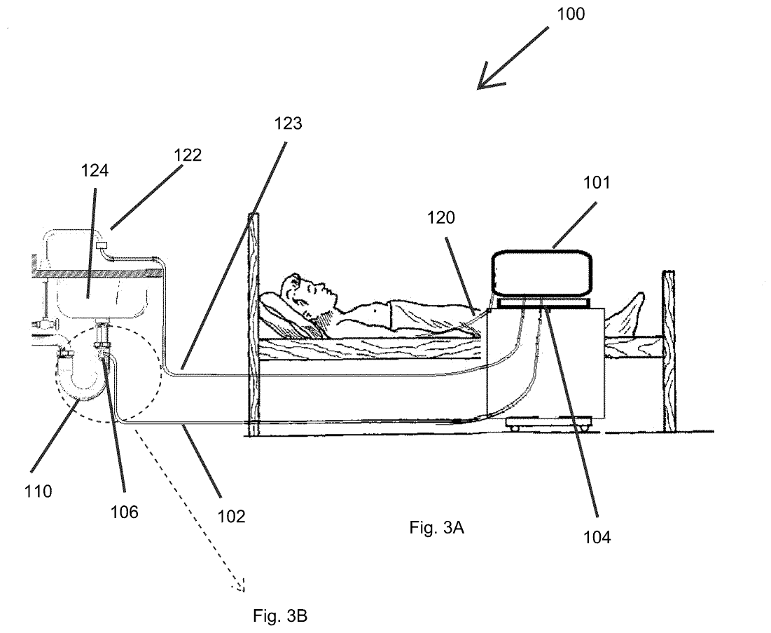

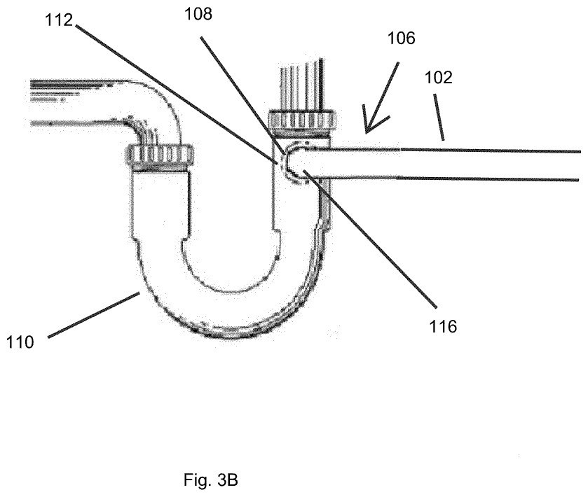

FIG. 1A is a side view of a plumbing trap, with an angled inlet 112 with receiving end 114, onto which a first quick connect 108 is affixed. FIG. 1B is a perspective view of the plumbing trap 110, also showing angled inlet 112, receiving end 114, and first quick connect 108. FIG. 1C shows the distal end 106 of conduit 102, and second quick connection 116. Second quick connection 116 will be connected to first quick connection 108, attached to angled inlet 112. Placement and operation of these elements are discussed further below, with respect to FIG. 3.

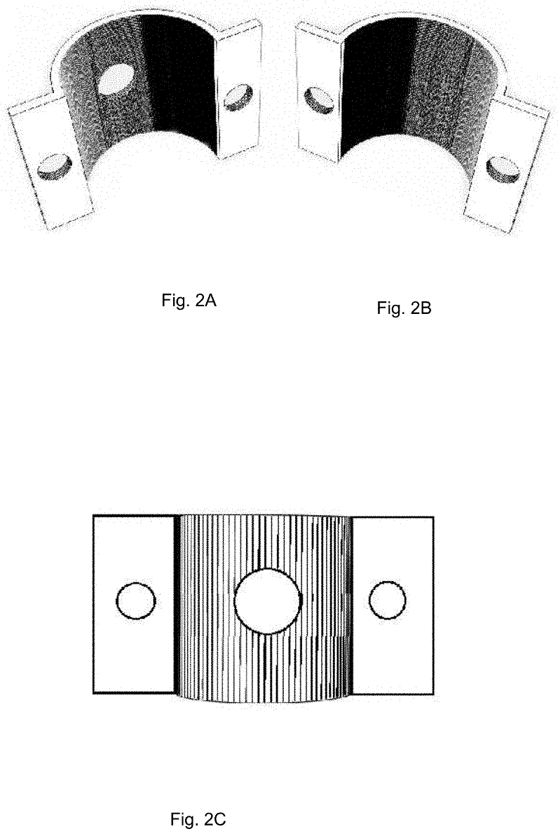

FIGS. 2A and 2B show two halves of a bracket used in old technology. In the old technology, a person would drill a hole into an existing plumbing trap for connecting draining lines from a home dialysis machine. The hole would be covered by the two halves when fastened by bolts or other means and forming an assembled bracket around the plumbing trap. The bracket holds the draining line end securely in the hole. When dialysis is no longer needed at the residence, the draining line would be disconnected, and the assembled bracket, still as two halves fastened, is turned so that the hole is covered. The bolts or other fasteners are then secured to hold the bracket as a seal over the hole. FIG. 2C is a front view of the two halves assembled and fastened by two bolts in holes shown (no reference numbers).

FIG. 3A shows the system 100 in operation. A patient may be in prone or sitting position, with dialysis tubing 120 connected to an arm and to a dialysis machine 101. Sitting is the most common position. Dialysis machine 101 takes a flow of blood from the patient, cleans it, and returns the cleaned blood to the patient. The dialysis machine 101 requires water, which typically is provided via a water line 123 connected to a water source 122. The waste removed from the blood by the dialysis machine 101 is conveyed by natural forces of gravity and air pressure from the dialysis machine 101, through a conduit 102, to the plumbing trap 110. The conduit 102 has a proximal end 104 connected to a port at the dialysis machine 101 for waste removal, and has a distal end 106 to be connected to plumbing trap 110. The plumbing trap 110 is deployed under a sink 124, and to pipelines connected to a municipal sewer system. A second quick connect 116 on the distal end 106 of the conduit 102 makes a secure connection to a first quick connect 108 on the angled inlet 112, which in turn is on the plumbing trap 110. The angled inlet 112 is angled so that the flow of waste into the plumbing trap 110 is likely to go downward and not splash upward into the sink 124. However, angled inlet 112 in fact could have no downward angle.

FIG. 3B is a close-up view, showing the connection of conduit 102 distal end 106 to angled inlet 112, via first quick connection 108 and second quick connection 116.

Typically, when dialysis equipment and infrastructure is installed in a residential setting it remains for a long term of days, weeks, months, or years, until no longer needed or until replaced. When the equipment is removed, care must be taken not to spill the waste.

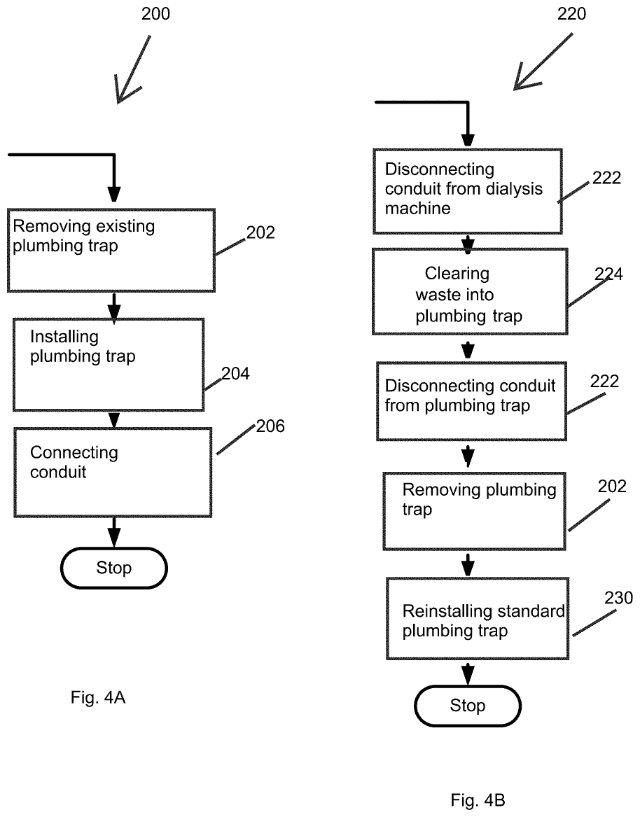

The subject technology, as a system and method, calls for long-term substitution of existing, standard plumbing traps with the plumbing trap 110. Use of the plumbing trap 110 eliminates the need to drill a hole in existing standard plumbing traps, and then having to cover the hole when dialysis is not needed. The method of turning the assembled bracket to cover the hole does not provide good seal, and is in effect a scar on the plumbing. The scar indicates that the residential plumbing system was used for medical waste. That information could cause loss of market value for the home. FIGS. 4A and 4B show, now, methods for implementing the system 100 that leave no scar.

FIG. 4A represents an install method 200, including the steps of removing 202 the existing plumbing trap, installing 204 plumbing trap 110, and connecting the conduit 102 to the angled inlet 112 via first quick connect 108 and second quick connect 116; and connecting conduit 102 at the proximal end to dialysis machine 101. At this point, after the patient is properly connected to the dialysis machine 101, the patient is properly situated (sitting or lying down), and the dialysis machine 101 is connected to water source 122 via water line 123, to power, and to all else as required, dialysis may proceed.

FIG. 4B represents a removal method 220, including the steps of disconnecting 222 the conduit 102 from the dialysis machine 101, clearing 224 waste into the plumbing trap 110, disconnecting 222 the conduit 102 from the plumbing trap 110, removing 202 the plumbing trap 110, and reinstalling 230 the original or replacement standard plumbing trap.

### ADVANTAGES

The system and method disclosed here provide several advantages, including:
a. Safe dialysis disposal into a sewer system
b. No permanent damage to existing plumbing
c. Low risk of residual leaks

While the foregoing written description enables one of ordinary skill to make and use what is considered presently to be the best mode thereof, those of ordinary skill will understand and appreciate the existence of variations, combinations, and equivalents of the specific embodiment, method, and examples herein. Unless claimed, particular system architecture and algorithms shown are not critical, but represent one or more embodiments.

I claim:

1. A system to conduct liquid medical waste into a sewer comprising,
a conduit having a proximal end and a distal end,
said proximal end being attached to receive liquid medical waste from a source,
said conduit having a first quick connection at said distal end;
a plumbing trap,
said plumbing trap having an angled inlet,
said angled inlet having a receiving end,
said angled inlet having a second quick connection at said receiving end; and
said conduit being connected to said plumbing trap through said first and said second quick connection to allow the liquid medical waste to drain into said plumbing trap.

2. A method of medical waste removal from a source to a sewer in a residential setting comprising,
removing an original plumbing trap;
installing a plumbing trap;
said plumbing trap having an angled inlet,
said angled inlet having a receiving end,
said angled inlet having a second quick connection at said receiving end;
connecting a conduit at the source to said angled inlet employing said first and said second quick connections;
disconnecting said conduit from said angled inlet when no longer needed;
removing said plumbing trap; and
installing said original plumbing trap.

3. The method of claim 2 wherein a replacement plumbing trap may be used in place of said original trap.
